(19) United States
(12) Patent Application Publication
(10) Pub. No.: US 2016/0113619 A1
(43) Pub. Date: Apr. 28, 2016

(54) STETHOSCOPE EARPIECE

(71) Applicant: Scott Gold, New York, NY (US)

(72) Inventor: Scott Gold, New York, NY (US)

(21) Appl. No.: 14/525,905

(22) Filed: Oct. 28, 2014

(51) Int. Cl. A61B 7/02; H04R 1/46

(52) U.S. Cl. CPC A61B 7/02; H04R 1/46

(57) ABSTRACT

Conically-shaped earpieces with flexible covers for a stethoscope are described, which sit on the outside of the user's ears and are particularly useful for users with hearing aids. A stethoscope structure with conically-shaped earpieces is also disclosed. The stethoscope structure has a branched elongated tube, ear tubes, a connector, a chest piece, at least one conically-shaped earpiece, and flexible cover for each earpiece.

### FIELD OF THE INVENTION

The present invention relates to earpieces for stethoscopes and, more particularly, to conically-shaped earpieces for stethoscopes that sit on, rather than within, the ear.

### BACKGROUND OF THE INVENTION

Stethoscopes are important tools for health care professional to evaluate and diagnose patients. They have been used in the medical profession since the 1800s. The design, as well as the functionality, of medical stethoscopes is well-documented in the art. Typically, stethoscopes have a substantially U-shaped or heart-shaped design, with earpieces that sit within the user's ear canal to transmit sound. Examples of such stethoscope configurations are disclosed in U.S. Pat. No. 257,487 and U.S. Pat. No. 3,730,290.

According to www.hearingloss.org, about 20% of adults, which represents 48 million people, report some degree of hearing loss. At age 65, one out of three people have hearing loss. Thus, there are many individuals in the medical profession that have hearing loss and use hearing aids to compensate for that issue. Because Americans are living and working longer, the number of individuals that are working in the medical profession that use hearing aids is increasing. For health care professional with hearing aids, using a traditional stethoscope is difficult because of the requirement that the earpieces of the stethoscope sit within user's ear canal. Typical hearing aids include behind-the ear aids, in-the-ear aids, in-the-canal aids, and completely-in-the-canal aids. For each of these types of hearing aids, using a traditional stethoscope is not practical because the user must remove his or her hearing aid prior to use. Requiring removal of a person's hearing aid is time consuming, potentially embarrassing for the user, and can negatively impact the user's ability to hear the biological sounds from the stethoscope. While amplified stethoscopes may increase the person's ability to hear from the stethoscope, it does not fully solve the problem of using a stethoscope with hearing aids. It would therefore be beneficial to provide earpieces for stethoscopes that sit on the outside of the ear, which can be used with a variety of hearing aid types.

Prior art stethoscope systems that have used headphones in conjunction with a traditional stethoscope, include the design disclosed in United States Patent Application Publication No. 2002/0129989. While the headphones in such systems sit on the ears, the stethoscope earpieces are still required to sit within the user's ear, extending through the headphone in this particular construction. Thus, such systems do not solve the problems faced by users with hearing aids.

Other prior art stethoscope systems have attempted to solve the problem of using a stethoscope with a hearing aid. For example, Cardionics markets the “E-Scope” product, which requires the user to input electronic headphones into the stethoscope, which does do not use specialized earpieces to transmit sound. Such a system is not desirable because it significantly changes the appearance of the stethoscope and can alert the patient to the user's hearing aid. Thus, there exists a need for earpieces that are discrete and less bulky than over-the-head headphones. Furthermore, an article by Westone, titled “Scope This Out!,” discloses various stethoscope earpieces that are designed for users with hearing aids. These earpieces, however, bear against the hearing aid, which can cause discomfort to the wearer. Therefore, it is desirable to have a stethoscope earpiece construction which addresses these and other issues.

The ear canal is a sensitive part of the human body. Sensitivity can range from mild dermatitis of the ear canal to chronic otitis externa. Individuals with skin conditions such as eczema or psoriasis affecting their ears are particularly susceptible to such inflammation. Irritation of the ear canal is also common among health care professionals, who often utilize stethoscopes with earpieces that sit within their ear canal. While using antibacterial or softer earpieces may reduce such irritation, it does eliminate the problem. Thus, to further reduce irritation to the ear canal, earpieces for a stethoscope that sit on the outside of the ear are desirable.

Stethoscopes can be shared among health care professionals, and not all stethoscope users will require earpieces that sit on, rather than within, the ear. Therefore, it is desirable for the stethoscope earpiece to be removable.

It is with respect to these problems that the present invention is addressed.

### SUMMARY OF THE INVENTION

In accordance with one aspect of the present invention, an earpiece for a stethoscope is disclosed. The earpiece is configured to be connected to the ear tube of a stethoscope. The earpiece can be conically-shaped with an apex and a base, so as to define a sound chamber. The apex of the earpiece has a collar with a fitting, which is adapted to fit on the ear tube of a stethoscope. The base of the earpiece is sufficiently sized to sit on the surface of the user's ear and transmit sound from the ear tube of a stethoscope to the earpiece's sound chamber. Sound is then transferred from the earpiece's sound chamber into the user's ear. The earpiece further comprises a flexible cover, which is seated about the base′ of the earpiece. The flexible cover operates to engage and create a seal between the base of the earpiece and the user's ear when the earpieces of the stethoscope are biased towards each other.

In one aspect of the invention, the flexible covers can be made of foam. In accordance with still further aspects of the invention, the top end of the flexible cover has an opening with an annular access, which provides access to the space between the top and bottom end of the flexible cover. In such arrangements, the bottom of the annual access on the flexible cover provides a bearing surface for the earpiece. In various arrangements of the invention, the bearing surface can be textured and the annular access can be sealed to prevent degradation of the flexible cover at the annular access.

Optionally, the fitting of the collar of the earpiece can be removable from the ear tube of a stethoscope. Such fitting may be threaded, and attachable to complementary threading on the ear tube. Also optionally, the earpiece can further comprise a set of flanges, which are connected to either the earpiece or the ear tube. Such flanges are adapted to sit on the pinna of the ear to support the weight of the stethoscope when in use. Still further optional features include that the base of each conically-shaped earpiece can comprise an overlapping section that allows for the diameter of the base of each earpiece to be adjusted based on the size of the user's ear.

In another aspect of the invention, a plurality of interchangeable, preferably conically-shaped earpieces, as described above, are provided in an earpiece kit for use with a stethoscope. A plurality of flexible covers, as described above, is also provided.

In accordance with still a further aspect of the present invention, a stethoscope structure is disclosed. The stethoscope structure includes a branched flexible tube, a pair of rigid ear tubes that are coupled in fluid communication with the branching portion of the flexible tube, a connector at the lower ends of the ear tubes for biasing the ear tubes towards each other to define a substantially U-shaped assembly, a chest piece, at least one preferably conically-shaped earpiece, as described above, and at least one flexible cover, as described above.

These and other aspects, features and advantages shall be apparent from the accompanying drawings and description of certain embodiments of the invention.

### BRIEF DESCRIPTION OF THE DRAWINGS

FIG. 1 is a perspective view of the earpiece with the flexible cover, according to one embodiment of the invention;

FIG. 2 is an exploded perspective view of the earpiece with the flexible cover;

FIG. 3 is a front elevation view of a stethoscope with the earpieces with the flexible covers, according to the first embodiment;

FIG. 4 is a top elevation view of the flexible cover;

FIG. 5 is a top elevation view of the earpiece of the first embodiment with the flexible cover; and

FIG. 6 is a top elevation view of the earpiece, according to a second embodiment showing the flexible cover with a flange;

### DETAILED DESCRIPTION OF CERTAIN EMBODIMENTS OF THE INVENTION

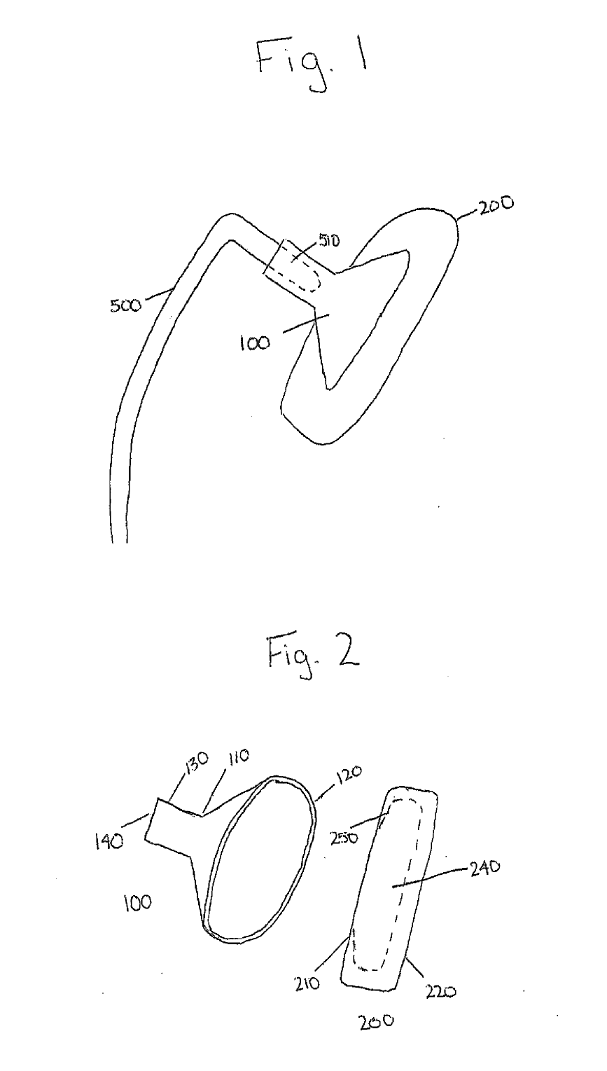

Referring to FIGS. 1 and 2, the earpiece configuration in the embodiment as illustrated, comprises preferably a conically-shaped earpiece 100 with an apex 110 and a base 120. As shown in FIG. 2, the earpiece 100 is open at both the base 120 and the fitting 140 of the collar 130 at the apex 110. The fitting 140 of the collar 130 is adapted to fit over the ear tube of a stethoscope. In the preferred embodiment, the collar 130 is cylindrical and connected to the earpiece cone at the apex 110. The base 120 of the earpiece 100 is sufficiently sized to fit an adult ear. This earpiece configuration, as shown in FIGS. 1 and 2, allows for the ear tube 500 of the stethoscope to be away from, rather than within, the ear drum such as to allow room for a hearing aid in the ear. In one embodiment, the base 120 of the earpiece is approximately 2 inches in diameter. Because of the small size of the earpiece 100 and flexible cover 200, health care professionals can use the present invention discretely. In a further embodiment, the conically-shaped earpiece 100 is a flexible rubber. Rubber is preferred because it is flexible and can be manipulated to fit over differently sized ear tubes. However, it is appreciated that the earpiece 100 can be made out of any suitable material.

The conical shape of the earpiece 100 is an important aspect of the invention. It defines a sound chamber, which transmits, directs, and amplifies sound to the user's ears. Similar to a megaphone, the conical shape of the earpiece 100 amplifies sound by directing the sound waves from the stethoscope's ear tube 500 to the user's ear. The conical shape of the earpiece 100 is further preferred because it allows for space between the ear tube 500 of the stethoscope and the user's hearing aid. The earpiece may have other shapes, such as hemispherical, but these are less preferred as it provides a sound chamber which is less efficient.

Either one or two earpieces can be used on the stethoscope, depending on the user's needs. For example, if a user only has a hearing aid in the right ear, the earpiece disclosed can be used on the right ear tube of the stethoscope while a traditional earpiece can be used on the left ear tube of the stethoscope.

Another important aspect of the invention is the flexible cover 200 for the earpiece 100. Referring to FIG. 1, the flexible cover 200 is seated about the base 120 of the earpiece 100. The flexible cover 200 wraps around the base 120 of the earpiece such that it provides a generally sealing engagement between the base of the earpiece 100 and the user's ear. The flexible cover 200 is made from a flexible material so that the user can cover and uncover the earpiece easily. The flexible cover 200, however, must be sufficiently durable to withstand tearing from such use. In one embodiment, foam is used for the flexible cover 200. Foam is the preferred material for the flexible cover 200 because it is flexible, durable, inexpensive, and provides a cushion between the earpiece and the user's ear. The ability of the foam to provide a cushion between the earpiece and the user's ear is important, because other prior art earpieces that can be used with hearing aids lack this feature and can cause user discomfort. Foam is further preferred because it has a higher coefficient of friction, thus reducing the problem of the earpiece 100 slipping off of the user's ear. Other materials, such as padded leather or rubber may also be used. It is appreciated that any ear cushion or ear pad used in the art for headphones may also be suitable for the flexible cover 200 in the present invention. The flexible cover 200 is further advantageous because it disguises the conically-shaped earpiece and covers the majority of the user's ear during use, providing a seal over the ear and any part of the hearing aid which may extend outside of the ear (e.g., over-the-ear hearing aid types). This reduces the chance that a patient will be alerted to the fact that the health care profession has a hearing aid.

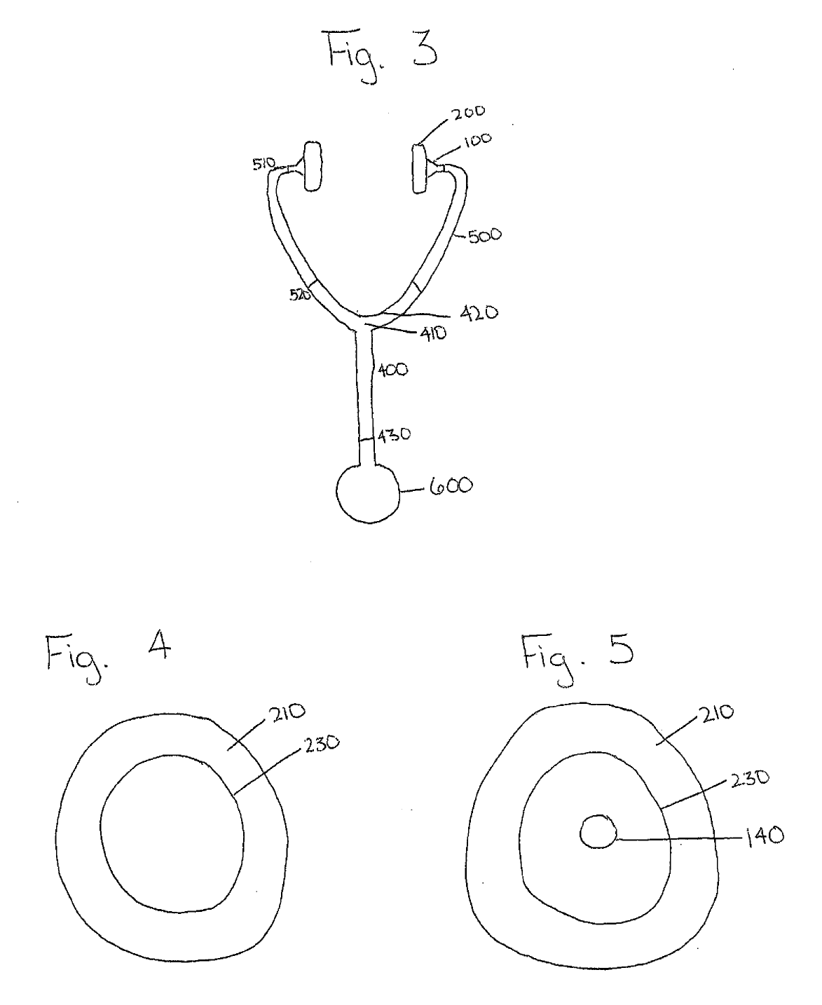

In one embodiment, the flexible cover has an opening with an annular access 230 on its top end 210, as shown in FIGS. 2, 4 and 5. The annular access 230 provides access to a space 240 between the top 210 and bottom end 220 of the flexible cover 200. The bottom of the annular access 250 provides a bearing surface for the earpiece 100. In one embodiment, the bearing surface is textured to prevent the flexible cover 200 from sliding off of the earpiece 100. The texturing can consist of rubber bumps or another material with a high coefficient of friction. In another embodiment, the annular access 230 is sealed at its edges to prevent degradation of the flexible cover. Because the earpiece 100 is covered and uncovered through the annular access 230, the flexible cover 200 could fray or rip at the annular access 230. Sealing the edge of the annular access 230 prevents such degradation.

For purposes of sharing stethoscopes, it is advantageous to have an earpiece kit comprising a plurality of interchangeable, conically-shaped earpieces 100, as described above. A plurality of flexible covers 200 would also be included, and each would be adapted to cover its respective earpiece 100. In another aspect of the invention, the diameter of the base 120 of the earpiece 100 can be adjusted to fit a variety of ear sizes. This allows for manufacturers to create one earpiece mold, thus reducing costs. Healthcare professionals can also share the adjustable earpiece 100, because it can be resized to properly fit each user. It is recommended that each user cover the adjustable earpiece 100 with his or her own flexible cover 200 to prevent the spread of disease. The base 120 of the earpiece 100 consists of an overlapping section, which allows for the diameter of the base to be increased or decreased. The desired diameter can be secured by any suitable method, such as a clip or locking mechanism on the earpiece.

The fitting 140 of the collar 130 can be attachable and detachable from the ear tube 500 of the stethoscope. The removable functionality of the earpiece configuration is advantageous because it allows for multiple healthcare professionals to use the same stethoscope with his or her own earpieces. This reduces the transfer of bacteria and decreases the risk of getting an ear infection. The removability of the earpiece 100 is further beneficial because it allows for this invention to be used in conjunction with most stethoscopes on the market today. Because the fitting 140 is preferably made from a flexible rubber, it can adapt to fit a variety of stethoscope ear tube sizes. The interior of the fitting 140 can be threaded, and is connectable to a stethoscope ear tube that has complementary threading.

Because the earpiece is sitting on, rather than within, the ear, it may be desirable to have a flange 300 that supports the weight of the stethoscope. The flange can be connected to either the curved end of the ear tube 500, which is near the user's ear during operation of the stethoscope, or on the earpiece 100. FIG. 6 shows an embodiment of the invention where the flange 300 is connected to the earpiece 100. The flange is of a suitable shape and size to sit on the pinna of the ear to support the weight of the stethoscope. The flange can be curved to sit on the top of the ear, behind the upper ear lobe, where the ear connects to the user's skull. Using the flange 300 is particularly beneficial for those with in-the-ear, in-the-canal, and completely-in-the-canal hearing aids. Alternatively, each flange is a suitable size and shape to be connected to a headpiece, which is designed to sit on the top of the head and support the weight of the stethoscope. In this configuration, each flange can be connected to either the curved end of the ear tube or the earpiece at one end of the flange, and the headpiece at the other end of the flange. The headpiece is preferably made of a thin material that is sufficiently durable to hold the weight of the stethoscope, such as wire, thin plastic, or fishing line. The headpiece is preferably thin so as to disguise it from patients during use. The aforementioned flanges can be removable, giving the user the option to use such features instead of relying on the biasing of the ear tubes towards one another to hold the weight of the stethoscope.

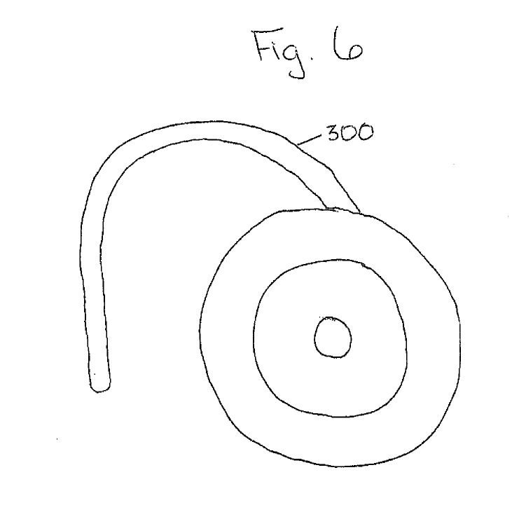

A stethoscope structure with preferably conically-shaped earpieces can be constructed with the inventive earpiece. Referring to FIG. 3, the stethoscope structure comprises an elongated flexible tube 400, which is branched in a “Y” configuration; a pair of rigid ear tubes 500 with an upper end 510 and a lower end 520, which are in fluid communication with the branched portion 420 of the flexible tube 400 and where the upper ends 510 of each ear tube are curved towards each other; a connector 410, preferably a spring connector, which is integrally attached to the lower end 520 of the ear tubes for biasing, or moving, the ear tubes towards each other to define a substantially U-shaped configuration with the ear tubes; and a chest piece 600, which is connected to the lower end 430 of the flexible tube that is opposite the branched portion. To this stethoscope structure, at least one preferably conically-shaped earpiece 100, as described above and shown in FIGS. 1 and 2, is adapted to fit on the upper end 510 of each ear tube. Each earpiece 100 is covered by a flexible cover 200, as described above and shown in FIGS. 1 and 5, to provide a generally sealing engagement between the base of each earpiece and the user's ear. The sealing engagement is a result of the connector 410, such as a spring connector, being used to bias the ear tubes 500 towards one another.

While the invention has been described in connection with certain embodiments, it is defined by the claims that accompany this description and is not to be read as being restricted to any one embodiment thereof.

What is claimed is:

1. An earpiece structure configured for connection to a stethoscope of the type having a pair of ear tubes with upper and lower ends, comprising:
at least one conically-shaped earpiece, each earpiece having an apex and a base so as to define a sound chamber within an interior thereof, wherein each earpiece is provided with a collar with a fitting at the apex, wherein the collar is adapted to fit on the upper end of the ear tube, and wherein the base has a size sufficient to seat the earpiece substantially on a surface of a user's ear, whereby sound transmits from the ear tube within the sound chamber and out of the base into the user's ear; and
at least one flexible covers seated, respectively, about the base of each earpiece, wherein the flexible covers provide a generally sealing engagement between the base of each earpiece and a respective one of the user's ears as a result of the biasing of the ear tubes towards one another.

2. The earpiece structure of claim 1, wherein the fitting of the collar of the earpiece to the upper end of the ear tube is removable.

3. The earpiece structure of claim 2, wherein the fitting of the collar of the earpiece has threads and wherein the upper end of the ear tube has complementary threads, such that the fitting is attachable and removable from the ear tube using the threads.

4. The earpiece structure of claim 1, further comprising a pair of flanges connected to the curved end of each respective ear tube or each respective earpiece, and wherein the flanges are adapted to sit upon the pinna of the ear and support the weight of the stethoscope.

5. The earpiece structure of claim 1, further comprising a headphone and a pair of flanges, each flange having a first end connected to the headpiece and a second, opposite end connected to the curved end of each respective ear tube or to each respective earpiece.

6. The earpiece structure of claim 1, wherein each flexible cover has a top end and a bottom end, the top end of the flexible cover further comprising an opening with an annular access with a top and a bottom which provides access to the space between the top end and bottom end of the flexible cover, wherein the bottom of the annular access on the flexible cover provides a bearing surface for the earpiece.

7. The earpiece structure of claim 6, wherein the bearing surface is textured.

8. The earpiece structure of claim 1, wherein each flexible cover has a top end and a bottom end, such that the top end of the flexible cover further comprises an opening with an annular access which provides access to the space between the top end and bottom end of the flexible cover, wherein the annular access is sealed to prevent degradation of the flexible cover at the annual access.

9. The earpiece structure of claim 1, wherein the base of each conically-shaped earpiece further comprises an overlapping section allowing for the diameter of the base of each earpiece to be changed.

10. The earpiece structure of claim 1, wherein the earpiece is rubber.

11. An earpiece kit configured for connection of at least one earpiece to a stethoscope of the type having a pair of ear tubes with upper and lower ends, comprising:
a plurality of interchangeable, conically-shaped earpieces each having an apex and a base of different respective sizes so as to define respective sound chambers within an interior of each earpiece, wherein each earpiece is provided with a collar with a fitting at the apex, wherein the collar is adapted to fit on the upper end of the ear tube, whereby sound transmits from the ear tube within the sound chamber and out of the base into the user's ear; and
a plurality of flexible covers adapted, respectively, to sit around the base of each different size earpiece, wherein the flexible covers provide a generally sealing engagement between the base of each earpiece and one of the user's ears as a result of the biasing of the ear tubes towards one another.

12. A stethoscope structure comprising,
an elongated flexible tube having at one end a pair of branching tube portions;
a pair of rigid elongated ear tubes with lower ends and upper ends, wherein the lower ends are coupled in fluid communication with the branching portions, and wherein the upper ends are curved towards one another;
a connector integrally attached to the lower ends of the ear tubes and biasing the ear tubes towards each other to define a substantially U-shaped assembly together with the ear tubes;
a chest piece, wherein the chest piece is connected to the flexible tube at the end opposite the branching portions;
at least one conically-shaped earpiece, each earpiece having an apex and a base so as to define a sound chamber within an interior thereof, wherein each earpiece is provided with a collar with a fitting at the apex, wherein the collar is adapted to fit on the upper end of the ear tube, and wherein the base has a size sufficient to seat the earpiece substantially on a surface of a user's ear, whereby sound transmits from the ear tube within the sound chamber and out of the base into the user's ear; and
at least one flexible covers seated, respectively, about the base of each earpiece, wherein the flexible covers provide a generally sealing engagement between the base of each earpiece and a respective one of the user's ears as a result of the biasing by the connector.

13. The stethoscope structure of claim 12, wherein the fitting of the collar of the earpiece to the upper end of the ear tube is removable.

14. The stethoscope structure of claim 13, wherein the fitting of the collar of the earpiece has threads and wherein the upper end of the ear tube has complementary threads, such that the fitting is attachable and removable from the ear tube using the threads.

15. The stethoscope structure of claim 12, further comprising a pair of flanges connected to the curved end of each respective ear tube or each respective earpiece, and wherein the flanges are adapted to sit upon the pinna of the ear and support the weight of the stethoscope structure.

16. The stethoscope structure of claim 12, further comprising a headphone and a pair of flanges, each flange having a first end connected to the headpiece and a second, opposite end connected to the curved end of each respective ear tube or to each respective earpiece.

17. The stethoscope structure of claim 12, wherein each flexible cover has a top end and a bottom end, the top end of the flexible cover further comprising an opening with an annular access with a top and a bottom which provides access to the space between the top end and bottom end of the flexible cover, wherein the bottom of the annular access on the flexible cover provides a bearing surface for the earpiece.

18. The stethoscope structure of claim 17, wherein the bearing surface is textured.

19. The stethoscope structure of claim 12, wherein each flexible cover has a top end and a bottom end, such that the top end of the flexible cover further comprises an opening with an annular access which provides access to the space between the top end and bottom end of the flexible cover, wherein the annular access is sealed to prevent degradation of the flexible cover at the annual access.

20. The stethoscope structure of claim 12, wherein the base of each conically-shaped earpiece further comprises an overlapping section allowing for the diameter of the base of each earpiece can be changed.
